(19) United States
(12) Patent Application Publication
(10) Pub. No.: US 2024/0285266 A1
(43) Pub. Date: Aug. 29, 2024

(54) CHONDROPLASTY TOOL

(71) Applicant: Rowan University, Glassboro, NJ (US)

(72) Inventors: Erik C. Brewer, Conshohocken, PA (US); Jacob Mahon, Williamstown, NJ (US); Maura Francis, Philadelphia, PA (US); Brennen Covely, Pitman, NJ (US); Stephanie Fanok, Jackson, NJ (US); Frederick Hardenbrook, Marlton, NJ (US); Joseph Oteri, Atlantic Highlands, NJ (US); Samantha Struble, Vernon, NJ (US); Sean McMillan, Mullica Hill, NJ (US); Alfred Intintoli, West Chester, PA (US); Angelo Patrinicola, Philadelphia, PA (US)

(73) Assignees: Rowan University, Glassboro, NJ (US); Trice Medical, Inc., Malvern, PA (US)

(21) Appl. No.: 18/502,534

(22) Filed: Nov. 6, 2023

Related U.S. Application Data
(63) Continuation of application No. 16/851,913, filed on Apr. 17, 2020, now abandoned.
(60) Provisional application No. 62/835,202, filed on Apr. 17, 2019.

(51) Int. Cl. A61B 10/02; A61B 17/00; A61B 17/32; A61B 17/56

(52) U.S. Cl. CPC A61B 10/025; A61B 17/320016; A61B 2017/00424; A61B 2017/320064; A61B 2017/564

(57) ABSTRACT

A device for harvesting cartilage from a subject in a minimally invasive manner, and methods of use thereof, are provided.

### CROSS-REFERENCE TO RELATED APPLICATION

The present application is a continuation of, and claims priority to, U.S. patent application Ser. No. 16/851,913, filed Apr. 17, 2020, which claims priority under 35 U.S.C. § 119(e) from U.S. Provisional Patent Application No. 62/835,202, filed Apr. 17, 2019. The disclosures of which are hereby incorporated by reference herein in their entireties.

### FIELD OF INVENTION

The invention pertains to methods, apparatus, and systems for performing minimally invasive method of harvesting live cartilage from the knee.

### BACKGROUND OF THE INVENTION

Articular cartilage damaged through acute or chronic trauma or osteochondritis dessecans, has limited ability to regenerate, leading to the symptoms of pain, restricted mobility and locking. Current treatment methods to stimulate repair of the cartilage include shaving the margins of the damaged cartilage to remove mechanical obstructions or irritants (abrasion or debridement) or drilling through the cartilage through the underlying bone into the vascular marrow in order to permit the ingrowth of fibrocartilage from the marrow. Long-standing severe damage to the articular cartilage can lead to debilitating osteoarthritis, which ultimately may require a total knee arthroplasty. In the United States, roughly six million people are affected by damaged articular cartilage in the knee.

Autologous chondrocyte implants (autologous chondrocyte transplant) has been investigated as a less invasive means for repairing cartilage defects in the knee. However, this procedure requires two arthrotomy surgeries spaced 6-weeks apart: one for harvesting healthy cartilage cells and another to implant the cultured chondrocytes into the cartilage defect. It also has a recovery time of 6-8 weeks. The first invasive procedure requires general anesthesia and a surgical preparation of the area. The surgeon then makes an incision to the area and uses a tool to cut the cartilage. Then, a second forceps tool is used to and retrieve the amount of cartilage that was cut. The invasiveness of the procedure has drawbacks and risks that are associated with surgery, as well as an extended recovery time.

Three of the four components of autologous chondrocyte implantation—use of a periosteal flap, debridement and rehabilitation—are not unique to autologous chondrocyte implantation, and these components of the procedure may account for some or all of the clinical improvements noted in uncontrolled studies of this procedure. Whether due to improvement of symptoms resulting from the three components or patients opting not to experience a second open arthrotomy, less than 30% of patients who begin the treatment protocol have the second open arthrotomy and as a result do not have the implantation of their cultured chondrocytes.

The forgoing examples of related art as to harvesting cartilage including customized instruments to the task, and limitations related therewith, are intended to be illustrative and not exclusive, and they do not imply any limitations on the invention described and claimed herein. Various limitations of the related art will become apparent to those skilled in the art upon a reading and understanding of the specification below and the accompanying drawings.

### SUMMARY OF THE INVENTION

One aspect of the invention provides a device for harvesting cartilage from a subject, wherein the device is configured to excise and harvest cartilage from the subject in a minimally invasive manner. The cartilage being harvested can be articular cartilage, removed from either the medial or lateral femoral condyle or the intercondylar notch.

This aspect of the invention can have a variety of embodiments. The device can be configured to be inserted into a subject via a cannula. The device can be configured to be inserted into a knee or a shoulder of the subject.

The device can comprise a lower gouge configured to excise cartilage away from a cartilage surface, and an upper jaw, which rotates about the lower gouge, wherein the upper jaw is configured to close down and grip the excised cartilage. The device can be further configured to retract the harvested cartilage back into the cannula for extraction. The device can also be configured to be actuated by a handle with a sliding thumb grip.

The device can be configured to be completely contained within the cannula. The cannula can have an outer diameter of about 2.77 mm. The device can provide about 200 mg of cartilage in 3-5 passes.

Another aspect of the invention provides a method of harvesting cartilage from a subject. The method includes: inserting into a knee or shoulder of a subject under local anesthesia and direct visualization a device comprising a lower gouge configured to excise cartilage away from a cartilage surface, and an upper jaw, which rotates about the lower gouge, wherein the upper jaw is configured to close down and grip the excised cartilage; and excising and harvesting a cartilage from the knee or shoulder of the subject in a single pass by actuating the device.

This aspect of the invention can have a variety of embodiments. The cartilage can be articular cartilage. The cartilage can be from the non-load bearing surfaces of the medial and lateral femoral condyles. The method cartilage is harvested for a future open arthrotomy procedure to implant the autologous chondrocyte. About 200 mg of cartilage can be harvested in 3-5 passes. The cartilage can be harvested for a clinical articular cartilage biopsy procedure.

The device can be inserted into the knee or shoulder of the subject via a cannula under local anesthesia and direct visualization. The method can further include retracting the excised cartilage back into the cannula for extraction.

The method can also further include administering local anesthesia to the subject. The cartilage can be harvested under direct visualization via minimally invasive in-office an arthroscopy surgery.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1A depicts a top view of a cartilage procurement device (Prototype 1) according to an embodiment of the present invention. FIG. 1B depicts a side view of the device shown in FIG. 1A. FIGS. 1C-1D depict images from validation testing of the device depicted in FIG. 1A. FIG. 1C depicts cartilage biopsy of swine ears. FIG. 1D depicts cartilage biopsy of bovine femoral condyle.

FIG. 2A depicts a perspective view of a cartilage procurement device (Prototype 2) according to an embodiment of the present invention, with the upper jaw closed down on the lower jaw. FIG. 2B depicts a perspective view of the device shown in FIG. 2A, with the jaws open. FIG. 2C depicts a side view of the device shown in FIG. 2A, with the upper jaw closed down on the lower jaw. FIG. 2D depicts a side view of the device shown in FIG. 2A, with the jaws open.

FIG. 3A depicts a perspective view of a cartilage procurement device (Prototype 3) according to an embodiment of the present invention. FIG. 3B depicts an exploded view of the device shown in FIG. 3A.

FIGS. 4A-4D depict several views of a gouge tip according to an embodiment of the present invention. FIG. 4A shows a perspective view of the gouge tip. FIG. 4B depicts a side view of the gouge tip. FIG. 4C depicts a front view of the gouge tip. FIG. 4D depicts a top view of the gouge tip.

FIGS. 5A-5D depict several views of a gripper according to an embodiment of the present invention. FIG. 5A shows a perspective view of the gripper. FIG. 5B depicts a side view of the gripper. FIG. 5C depicts a front view of the gripper. FIG. 5D depicts a top view of the gripper.

FIGS. 6A-6D depict several views of a holder according to an embodiment of the present invention. FIG. 6A shows a perspective view of the holder. FIG. 6B depicts a side view of the holder. FIG. 6C depicts a front view of the holder. FIG. 6D depicts a top view of the holder.

FIGS. 7A-7D depict several views of a link according to an embodiment of the present invention. FIG. 7A shows a perspective view of the link. FIG. 7B depicts a front view of the link. FIG. 7C depicts a top view of the link. FIG. 7D depicts a side view of the link.

FIGS. 8A-8D depict several views of a linkage according to an embodiment of the present invention. FIG. 8A shows a perspective view of the linkage. FIG. 8B depicts a side view of the linkage. FIG. 8C depicts a front view of the linkage. FIG. 8D depicts a top view of the linkage.

FIGS. 9A-9D depict several views of an attachment piece for the holder according to an embodiment of the present invention. FIG. 9A shows a perspective view of the attachment piece. FIG. 9B depicts a side view of the attachment piece. FIG. 9C depicts a front view of the attachment piece. FIG. 9D depicts a top view of the attachment piece.

FIGS. 10A-10D depict several views of a pin according to an embodiment of the present invention. FIG. 10A shows a perspective view of the pin. FIG. 10B depicts a side view of the pin. FIG. 10C depicts a front view of the pin. FIG. 10D depicts a top view of the pin.

FIGS. 11A-11D depict several views of a slider according to an embodiment of the present invention. FIG. 11A shows a perspective view of the slider. FIG. 11B depicts a side view of the slider. FIG. 11C depicts a front view of the slider. FIG. 11D depicts a top view of the slider.

FIG. 12A depicts non-load bearing areas of the condylar surface, where cartilage is harvested. FIG. 12B depicts a current excision method used in an open Autologous Chondrocyte Implantation procedure. FIG. 12C depicts current retrieval method used in an open Autologous Chondrocyte Implantation procedure.

FIG. 13 depicts an example of mosaicplasty treatment.

FIGS. 14A-14C depict testing of a cartilage procurement device (Prototype 3) on an open human cadaver knee on the non-load bearing regions for validation of its excising functionality.

FIGS. 15A-15B depict a close-up view of a device 300 according to an embodiment of the invention, showing the gripper 302 linked to the linkage 305 via the link 304. FIG. 15A depicts the device 300 with the gripper 302 oriented in the open position. FIG. 15B depicts the device with the gripper 302 closed down on the gouge tip 301.

FIGS. 16A-16B depict a view of a device 300 according to an embodiment of the invention, showing the gripper 302 linked to the linkage 305 and slider 308 via the link 304. FIG. 16A depicts the device 300 with the gripper 301 oriented in the open position. FIG. 16B depicts the device with the gripper 302 closed down on the gouge tip 301.

FIGS. 17A-17B depict various close-up views of a device 300 according to an embodiment of the invention, showing the link between the linkage 305, link 304, pin 307, and gripper 301. FIG. 17A depicts a close-up side view of the device 300 with the gripper 302 in the open position. FIG. 17B depicts a close-up angled view of the device 300 with the gripper 302 in the closed position and shows the gripper 302 positioned within the handle 303 via a rod 310 inserted into a slot 309 in the handle 303.

FIGS. 18A-18B depict side views of a device 300 according to an embodiment of the invention. FIG. 18A depicts a close-up side view of the device 300 with the gripper 302 in the closed position, showing the gripper 302 linked to the linkage 305 via the link 304 and pin 307. FIG. 18B depicts a side view of the device 300 with the gripper 302 closed down on the gouge tip 301, and shows the gripper 302 linked to the linkage 305 via the link 304 and pin 307.

FIGS. 19A-19B depict side views of a device 300 according to an embodiment of the invention. FIG. 19A depicts a side view of the device 300 with the gripper 302 in the closed position. FIG. 19B depicts a side view of the device 300 with the gripper 302 in the open position.

FIGS. 20A-20B depict top views of a device 300 according to an embodiment of the invention. FIG. 20A depicts a top view of the device 300 with the gripper 302 in the closed position. FIG. 20B depicts a top view of the device 300 with the gripper 302 in the open position.

### DETAILED DESCRIPTION OF THE INVENTION

Replacing the first open arthrotomy with a minimally invasive guided harvesting of the live cartilage should significantly increase patients' compliance with the treatment plan. A successful design would be a minimally invasive apparatus and method of harvesting live cartilage from the knee under local anesthesia and direct visualization. This is of upmost importance when transitioning procedures from the operating room to procedure rooms and office clinics.

Embodiments of the invention provide a variety of devices and methods for harvesting cartilage from a subject, in particular, for harvesting cartilage to be used in procedures for treating damaged articular cartilage.

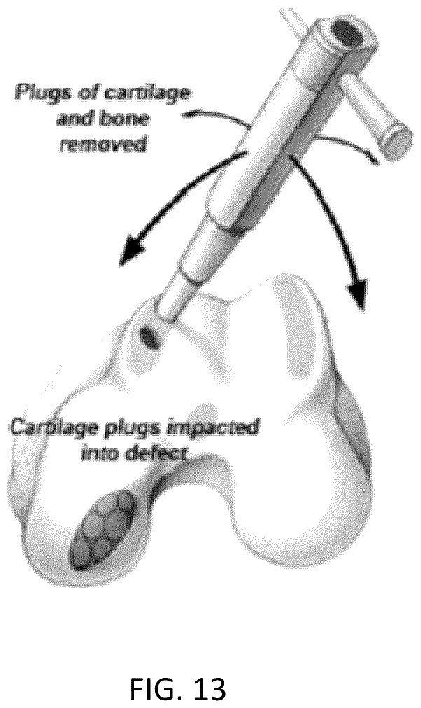

Current treatment available for damaged articular cartilage in the knee include Matrix-Induced Autologous Chondrocyte Implantation (MACI), debridement, marrow stimulation, and mosaicplasty surgery (FIG. 13). A MACI procedure requires two arthroscopic surgeries: one for harvesting healthy cartilage cells and another to implant the cultured chondrocytes into the cartilage defect. Embodiments of the invention eliminate the need for the first arthroscopic surgery in a MACI procedure, and replace the first surgery with a clinical procedure using a novel cartilage biopsy harvesting device described herein.

Embodiments of the invention provide chondroplasty tools that are useful, e.g., for a clinical cartilage biopsy procedure. One use of the device is to harvest an amount (e.g., about 200 mg) of articular cartilage from the patient that is required for the autologous chondrocyte implantation procedure in a clinical setting.

### Cartilage Procurement Devices

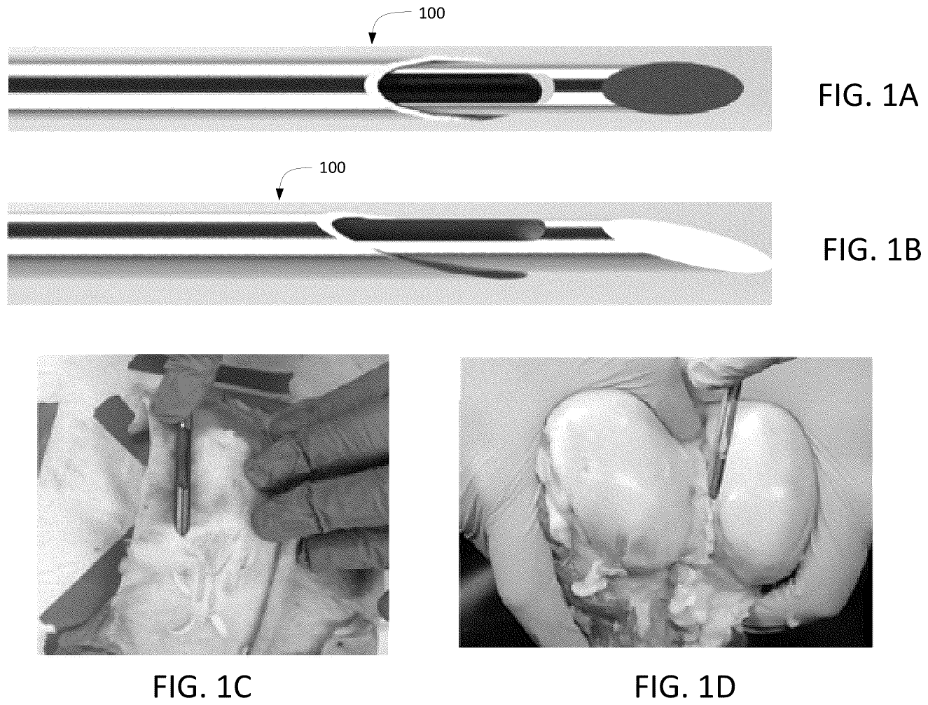

Referring now to FIGS. 1A-1B, one embodiment of the invention provides a cartilage procurement device 100 capable of cutting and harvesting cartilage in one pass (Prototype 1). The device 100 features a gouge design. The device 100 is capable of harvesting about 200 mg live cartilage. The device 100 is 12-gauge (2.77 mm outer diameter (OD)) and used with a cannula. The device 100 can be utilized with a local anesthetic, and in conjunction with a MI-EYE® camera enabled needle device.

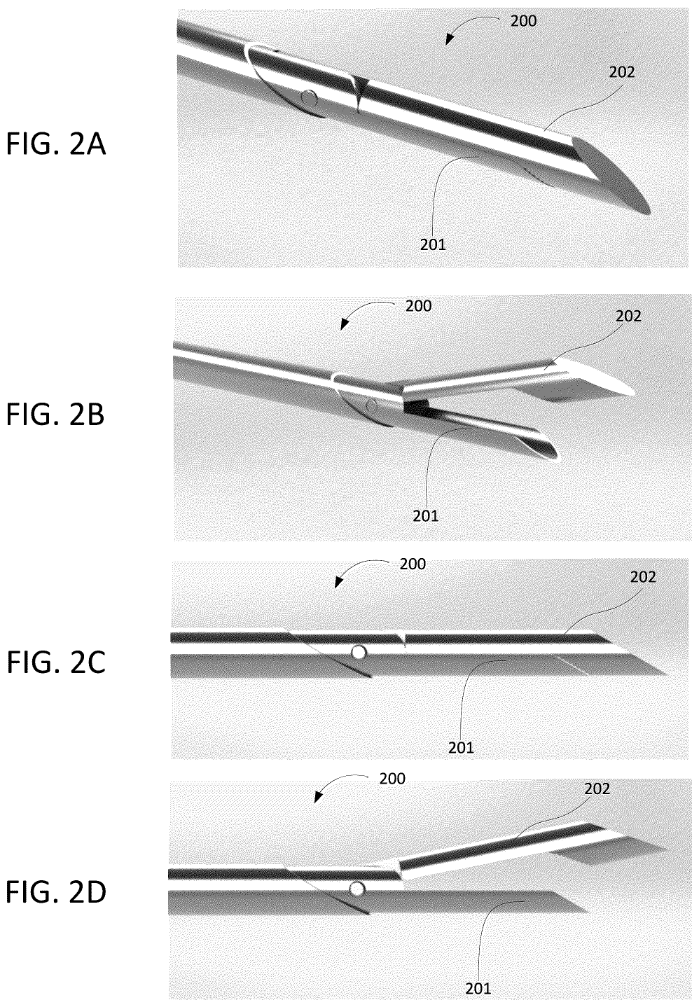

Testing, results and optimization of device 100 (Prototype 1) resulted in Prototype 2, shown in FIGS. 2A-2D. Referring now to FIGS. 2A-2D, another embodiment of the invention provides a cartilage procurement device 200 capable of cutting and harvesting cartilage in a single pass (Prototype 2). The device 200 includes a lower gouge jaw 201 that excises cartilage away from the articular cartilage surface. An upper herringbone jaw can act as a gripper 202, closing down on the excised cartilage, and pulling out from the condyle. The upper jaw 202 can then close and retract the biopsy procurement back into the cannula for extraction. A “pinless linkage” including a system of grooves and slots can enable the tool to be actuated by a handle with a sliding thumb grip. The design of Prototype 2 addresses issues of bending loads applied during cartilage excision, and instead distributes forces axially along its length. This gouge also features an articulating catchment gripper 202 to hold in the excised cartilage.

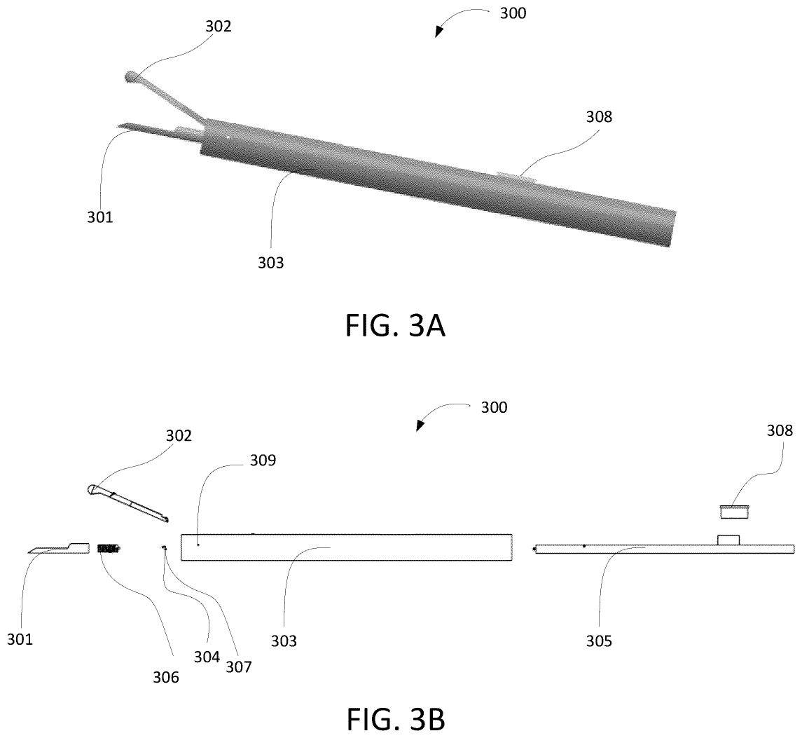

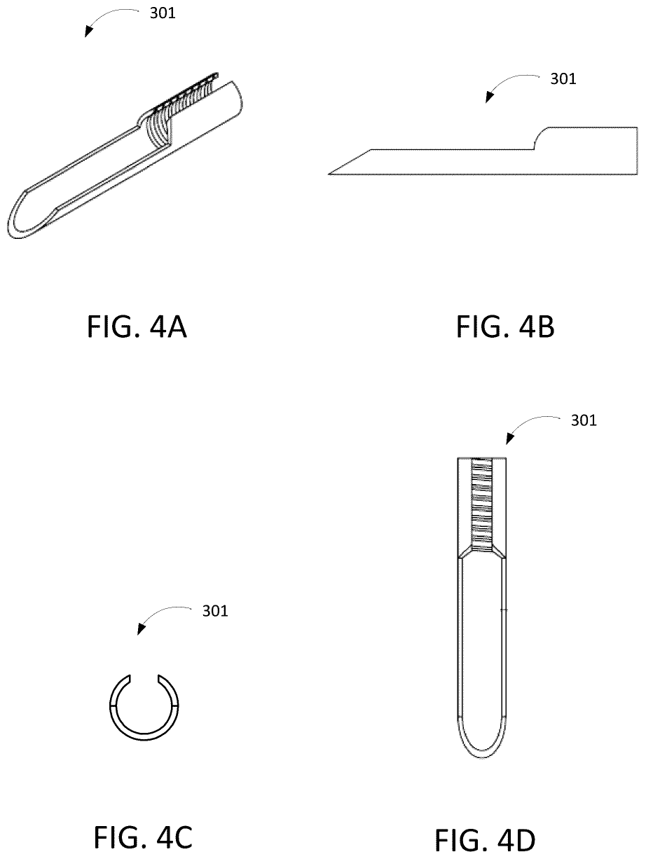

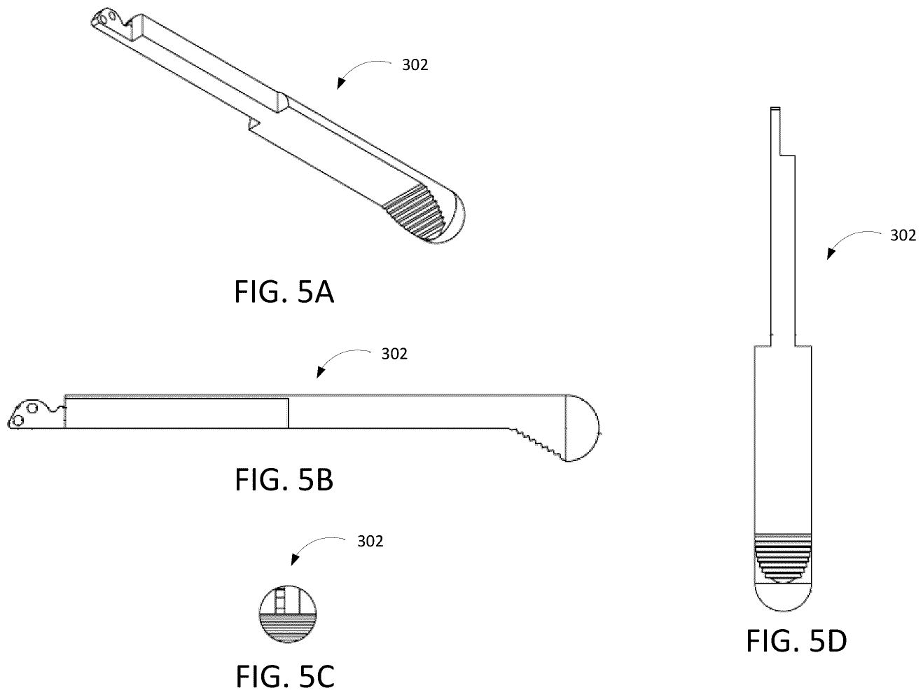

Referring now to FIGS. 3A-3B, another embodiment of the invention provides a cartilage procurement device 300 capable of cutting and harvesting cartilage in a single pass (Prototype 3). The device 300 can be inserted through a 12-gauge (2.77 mm) outer diameter cannula. The device 300 is designed to be 13-gauge (2.41 mm outer diameter). Once inside the medial or lateral compartments of the knee, the device 300 can protract outward. The device can include a lower gouge tip 301 (FIGS. 4A-4D) that is angled at 30-degrees that excises cartilage away from the articular cartilage surface. In one embodiment, the gouge tip 301 is made of stainless steel. An upper herringbone jaw can act as a gripper 302 (FIGS. 5A-5D), closing down on the excised cartilage, and pulling it out from the condyle. The gripper 302 can then close and retract the biopsy procurement back into the cannula for extraction. The design of Prototype 3 also addresses the issue of bending loads applied during cartilage excision and distributes the forces axially along its length. In one embodiment, the gripper 302 is made of stainless steel.

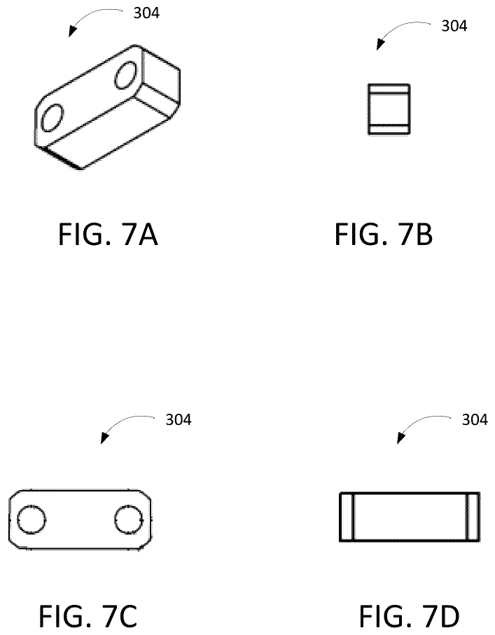

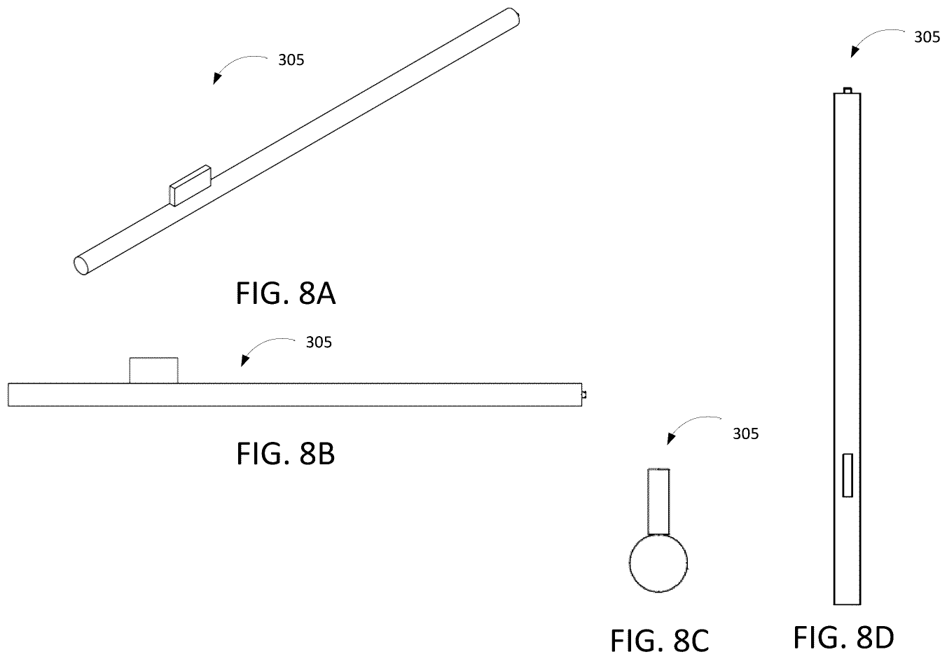

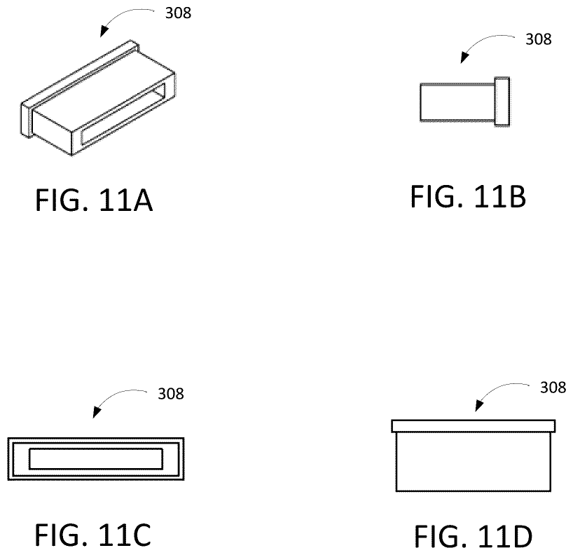

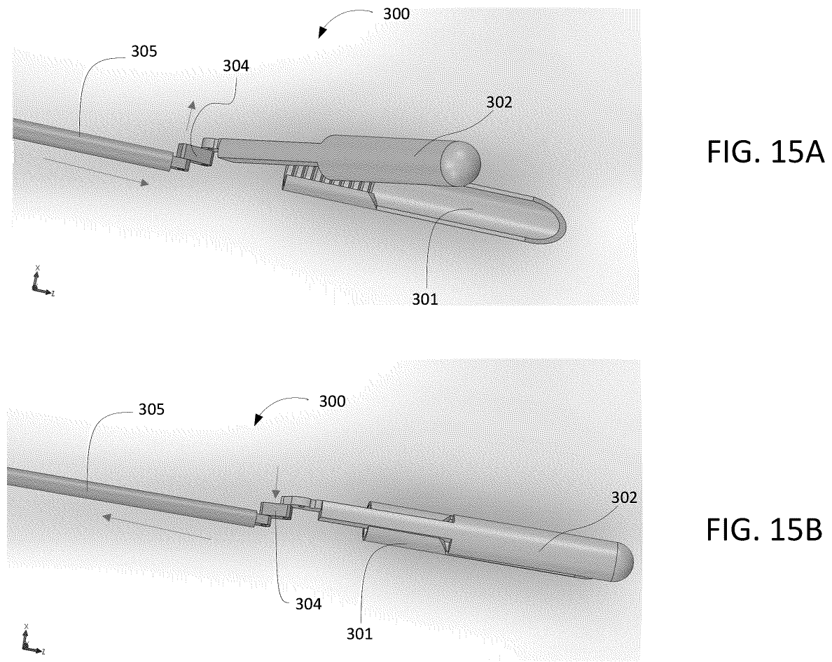

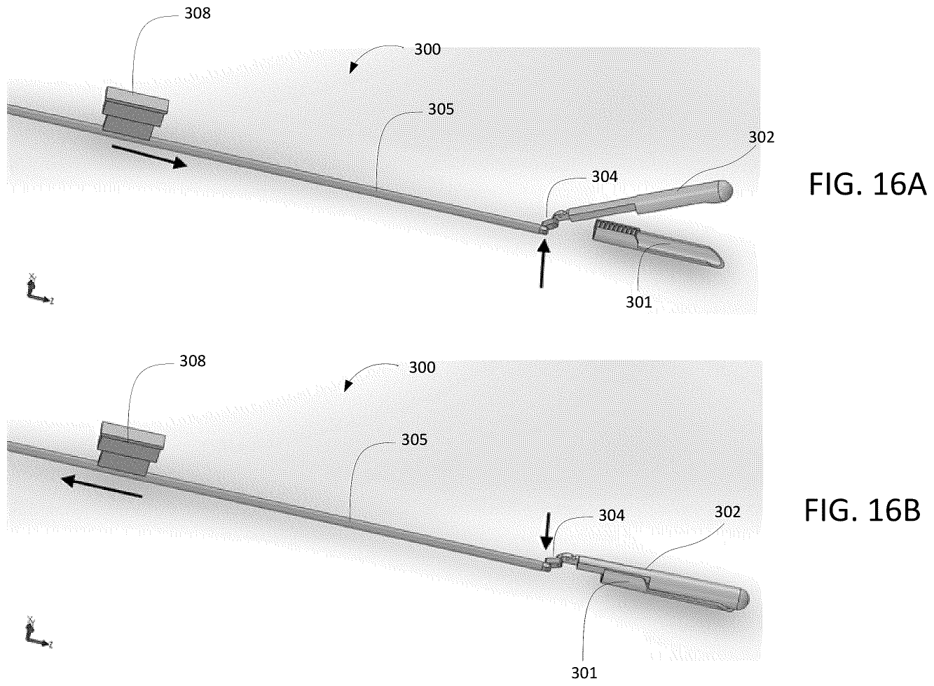

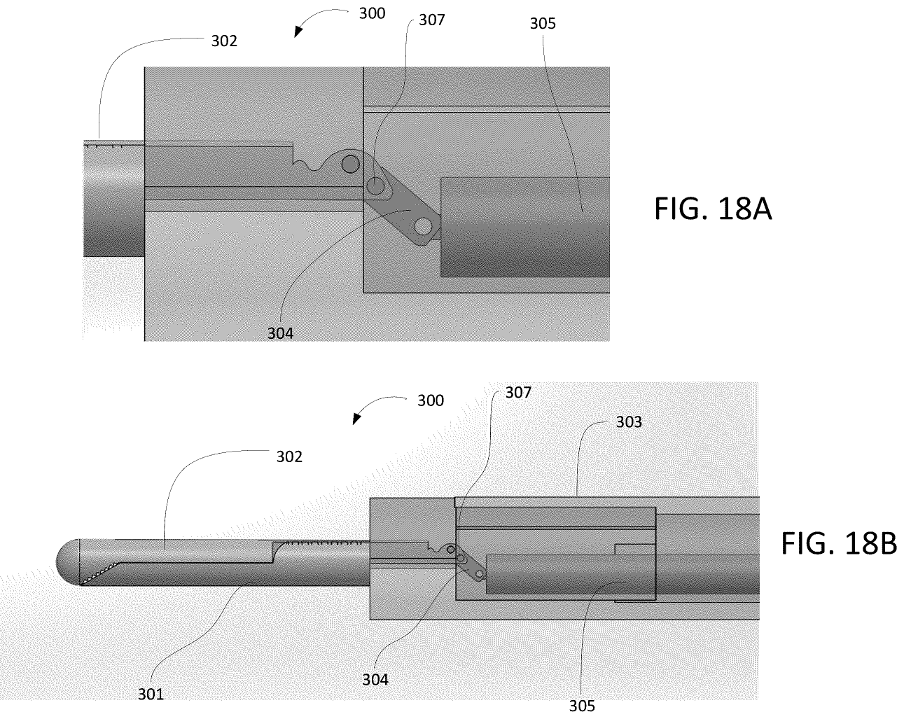

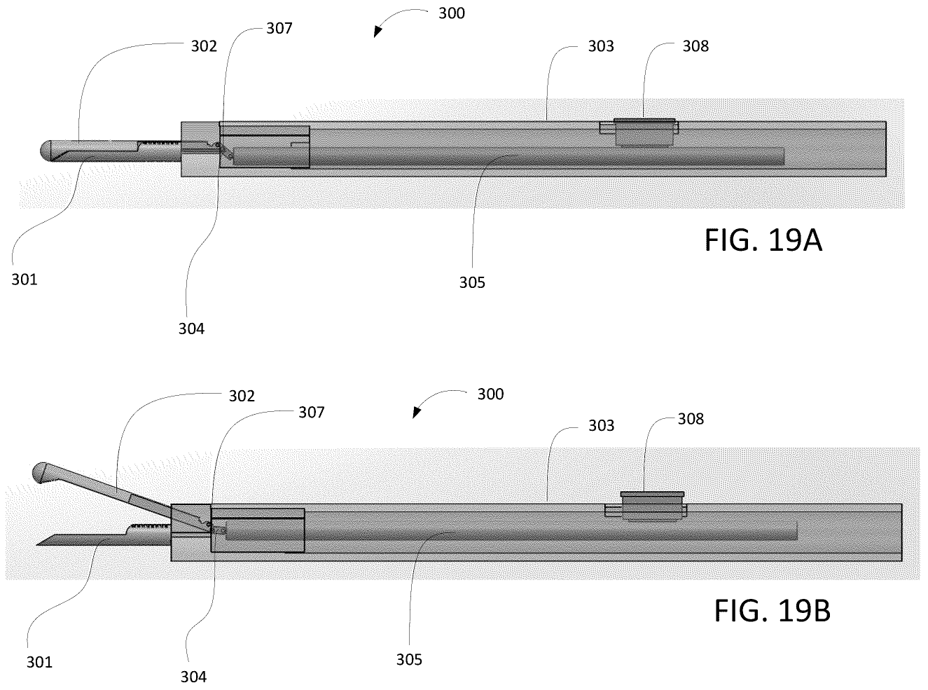

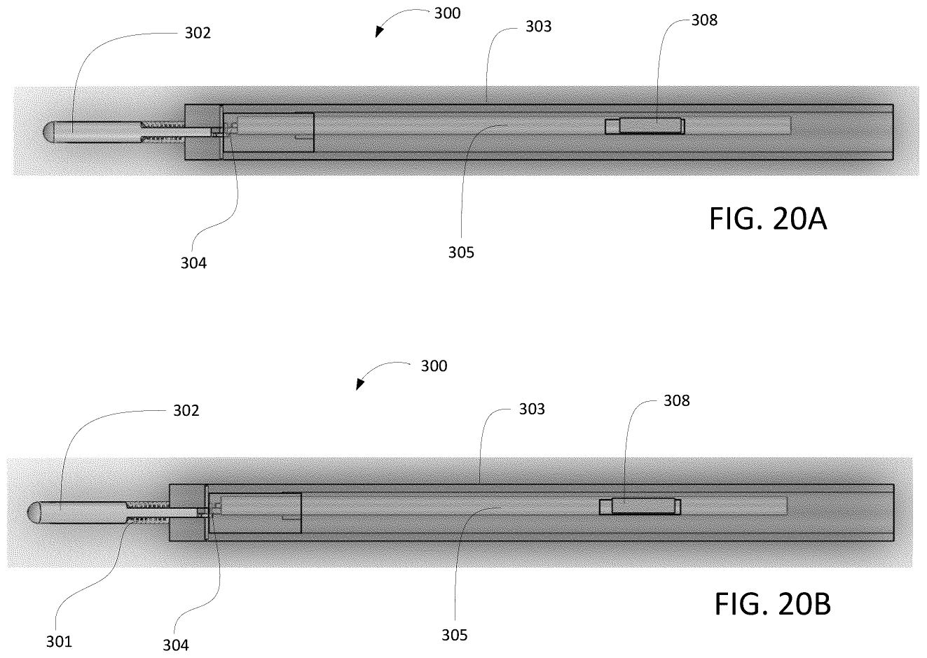

This design uses a linkage 305 (FIGS. 8A-8D) and link 304 (FIGS. 7A-7D) to allow the rotational motion of the gripper 301 so it can open and close. The slider 308 (FIGS. 11A-11D) attached to the linkage 305 allows the user to move the slider 308 back and forth while the gripper 302 opens and closes to obtain the cartilage during the surgery. As shown in FIGS. 15A-15B, 16A-16B, 17A-17B, 18A-18B, 18A-19B, and 20A-20B, when the slider 308 is moved back and forth, the link 304 rotates with the gripper 302 to rotate the gripper 302 up or down. When the slider 308 is moved, the link 304 shifts so the gripper 302 can open or close. In some embodiments, the gripper 302 can open up to 45° relative to the gouge tip 301. In some other embodiment, the gripper 302 can open up to greater than 45° relative to the gouge tip 301. In some embodiments, an opening can be made in the holder 303 to prevent the link 304 from hitting into the holder 303.

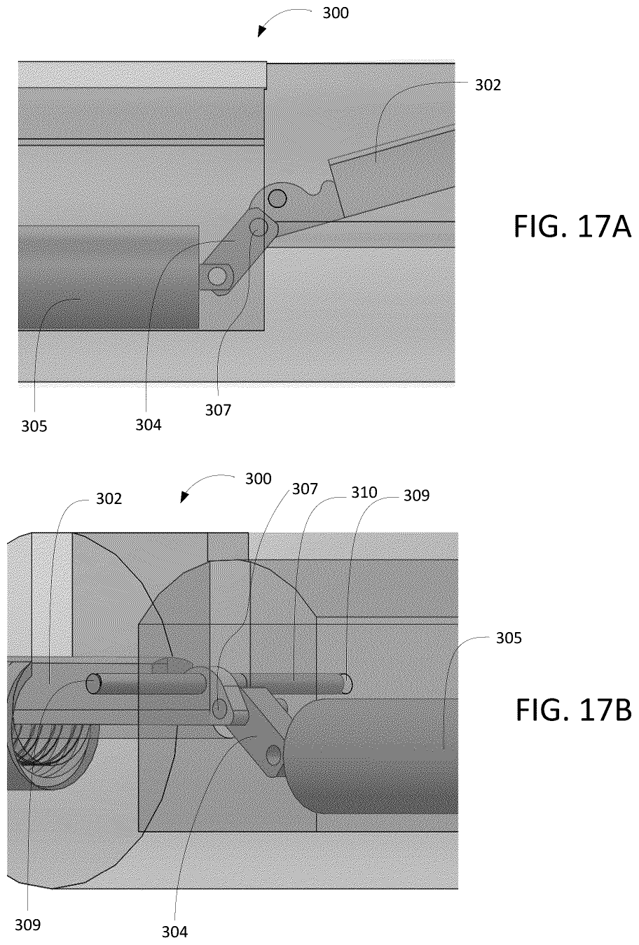

Referring now to FIG. 17B, the gripper 302 can be linked to the linkage 305 via a link 304 and pin 307. The gripper 302 can also connected to the holder 303 via a rod 310 inserted into a slot 309 in the holder 303. Movement of the linkage 305 shifts the position of the link 304, which then causes the gripper 302 to pivot about the rod 310, resulting into the gripper 302 rotating up or down to an open or closed position relative to the gouge tip 301.

In some embodiments, a spring or elastomer can be used to help with rotation of the gripper 302 or to add additional force to close the gripper 302. A locking mechanism can also be placed on the slider 308 to ensure there is no unnecessary movement during the procedure. In one embodiment, the linkage 305 is plastic. In one embodiment, the slider 308 is plastic.

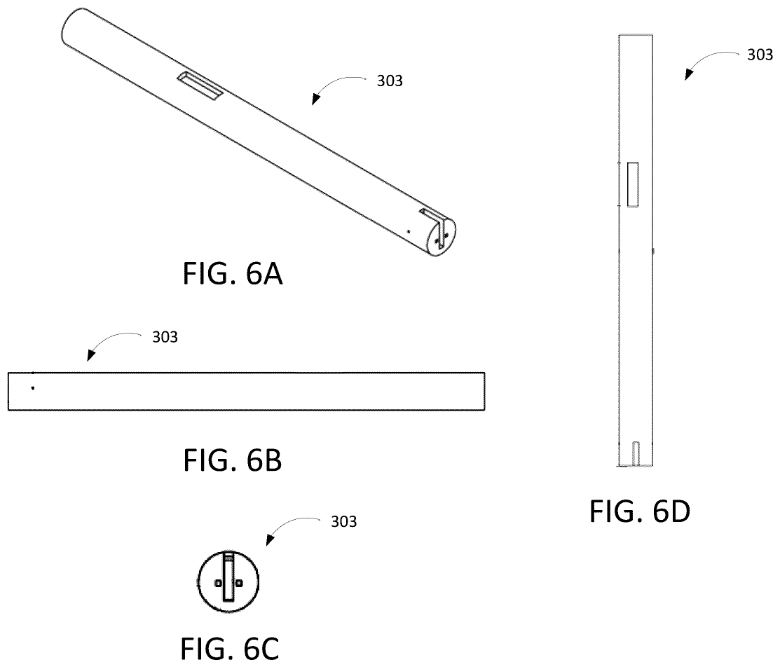

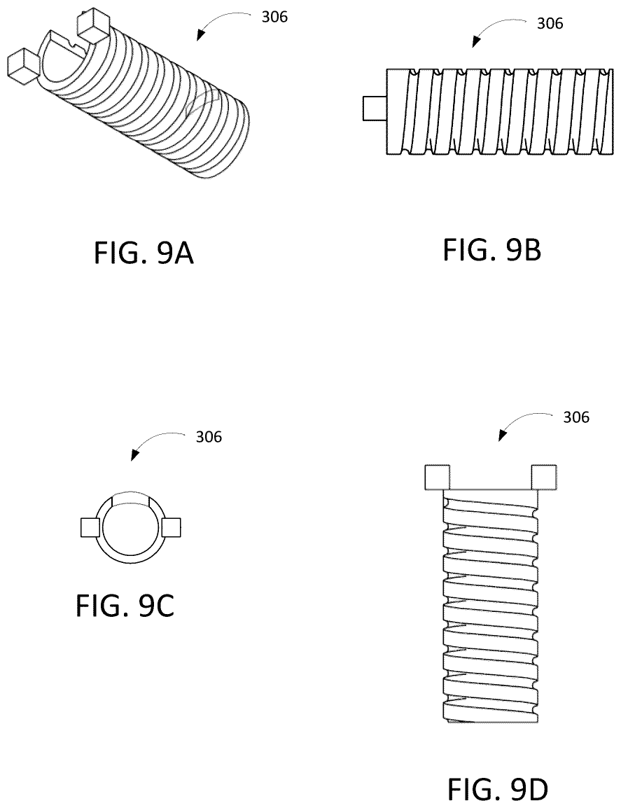

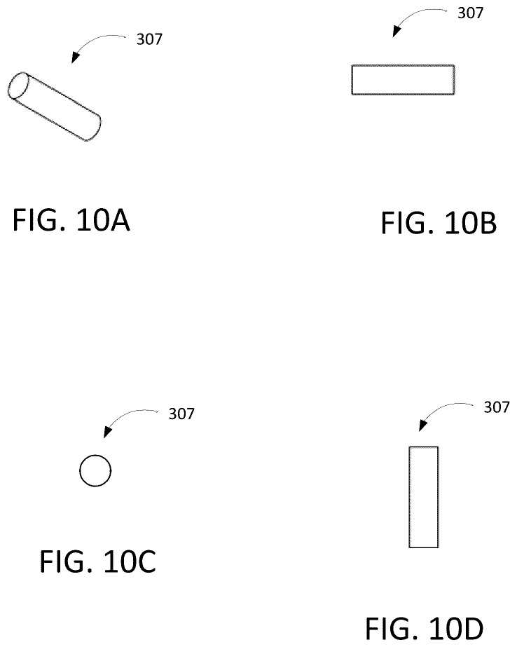

The lower gouge tip 301 can be threaded onto the handle portion 303 (FIGS. 6A-6D) of the device 300 via an attachment piece 306 (FIGS. 9A-9D), ensuring a snug fit. In one embodiment, the attachment piece 306 is made of stainless steel. In one embodiment, the handle 303 or holder 303 is plastic. The upper gripper 302 can be attached to a linkage arm 305 via a link 304 and a pin 307 (FIGS. 10A-10D). In one embodiment, the pin 307 is made of stainless steel. The pin 307 and linkage 305 can be inside the handle 303 of the device 300, ensuring no pin will be entering the body during a procedure. The linkage arm 305 can be actuated by a sliding thumb grip 308 on the handle 303.

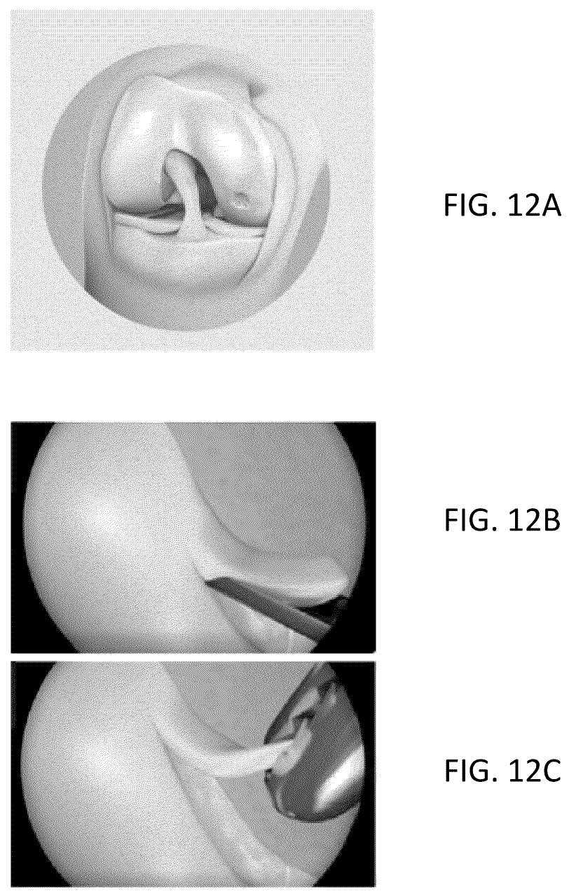

The device 100, 200, 300 can be inserted into the knee (e.g., via cannula) to harvest cartilage from the non-load bearing surfaces of the medial and lateral femoral condyles (FIG. 12A). The device 100, 200, 300 can be used for biopsy procurement of articular cartilage from the shoulder as well. This is the first device specifically designed for a clinical, articular cartilage biopsy procedure. The device 100, 200, 300 can be used in conjunction with an endoscope to procure biopsies from the condyles.

The device 100, 200, 300 can be completely contained with a 12-gauge (2.77 mm) outer diameter cannula, and thus, is an effective device for use in a clinical setting. Other devices and procedures are only used during open or endoscope-guided surgical arthroscopy. The device 100, 200, 300 is capable of providing 200 mg of articular cartilage in 3-5 passes. This is the mass required to generate effective cell cultures for a MACI procedure.

### Methods of Use

One embodiment of the invention provides a method of harvesting cartilage from a subject. The method utilizes a cartilage procurement device capable of excising and harvesting cartilage in a single pass, e.g., device 100, device 200, or device 300. As used herein, a “subject” may be a human or non-human mammal or a bird. Non-human mammals include, for example, livestock and pets, such as ovine, bovine, porcine, canine, feline and murine mammals. In certain embodiments, the subject is human.

The method can include the step of inserting a cartilage procurement device into a subject via a cannula. In one embodiment, the cannula is a 12-gauge (2.77 mm) outer diameter cannula. In one embodiment, the device is 13-gauge (2.41 mm outer diameter). In one embodiment, the cartilage procurement device is inserted into a knee or a shoulder of the subject via a cannula.

The method includes a step of excising and harvesting cartilage in a single pass. Once inserted into the knee or shoulder, the cartilage procurement device (e.g., device 100, device 200, or device 300) is actuated to excise and harvest cartilage in a single pass. In one embodiment, articular cartilage from the knee is excised and harvested in a single pass. In one embodiment, cartilage from the non-load-bearing surfaces of the medial and lateral femoral condyles is excised and harvested in a single pass. In another embodiment, articular cartilage from the shoulder is excised and harvested in a single pass.

In one embodiment, once the device is inserted into a knee (e.g., the medial or lateral compartments of the knee), the device 300 can protract outward. The device 300 includes a lower gouge tip 301 is used to excise cartilage away from the articular cartilage surface. Once cartilage is excised, the gripper 302 can close down on the excised cartilage and pull out the excised cartilage from the condyle.

The method can include the step of retracting the excised cartilage into the cannula for extraction. In one embodiment, the gripper 302 closes down on the excised cartilage and retracts the excised cartilage into the cannula for extraction.

In some embodiments, about 40 mg, about 50 mg, about 60 mg, or about 70 mg of cartilage is excised and harvested in a single pass. In one embodiment, the step of inserting the device 100, 200, 300 into the subject and/or the step of excising and harvesting cartilage in a single pass is repeated to harvest additional amounts of cartilage. For example, for a MACI procedure, about 200 mg of articular cartilage is required to generate effective cell cultures. In one embodiment, the method includes repeating the step of excising and harvesting the cartilage in single pass once, twice, three times, four times, or five times. In one embodiment, articular cartilage is harvested in 3 passes, 4 passes, or 5 passes. In one embodiment, about 200 mg of articular cartilage is harvested in 3-5 passes.

The method can be used in biopsy procedures, in particular, in a clinical articular cartilage biopsy procedure. The procedure can be part of a treatment of damaged cartilage, such as a treatment for damage articular cartilage, as described, for example, in “Cartilage Repair” University of San Francisco Health, https://www.ucsfhealth.org/treatments/cartilage-repair. In some embodiments, the device 100, 200, 300 is used in conjunction with other tool(s) used in biopsy procedures. In one embodiment, the device in conjunction with an endoscope to procure biopsies from the condyles.

In one embodiment, the method is used in a Matrix-Induced Autologous Chondrocyte Implantation (MACI) procedure. An exemplary MACI process is described in “A Step-By-Step Guide to the MACI Process.” MACI. www dot maci dot com/patients/how-maci-works/from-biopsy-to-surgery dot html. A MACI procedure requires two arthroscopic surgeries: one for harvesting healthy cartilage cells and another to implant the cultured chondrocytes into the cartilage defect. FIG. 12B shows a current excision method used in MACI, and FIG. 12C shows a current retrieval method used in MACI.

In one embodiment, the method using a cartilage procurement device capable of excising and harvesting cartilage in a single pass (e.g., device 100, device 200, or device 300) is used to harvest cartilage to be used to generate a cell culture for a MACI procedure. In one embodiment, the method eliminates the need for the first arthroscopic surgery in a MACI procedure. In one embodiment, the method replaces the first surgery with a clinical procedure using the cartilage procurement device described herein (e.g., device 100, device 200, or device 300).

In one embodiment, the method further comprises administering a local anesthesia to the subject (e.g., a local anesthesia to the knee or shoulder of the subject). In one embodiment, cartilage is excised and harvested from the subject without the need for an arthroscopic surgery. In some embodiments, the subject has damaged cartilage (e.g., damaged articular cartilage in the knee) in need of repair.

### EXAMPLES

### Example 1: Validation of Prototype 1

Swine ear cartilage was excised via retraction of the device 100 along a planar cartilage surface (FIG. 1C). Bovine cartilage was also excised using retraction along the femoral condyles (FIG. 1D). The device 100 was assessed for cartilage volume procured per pass of gouge, the time to obtain a 200 mg sample, and ergonomics and design feasibility.

### Prophetic Example 2: Validation of Prototype 2

Device 200 (Prototype 2) is tested on a bovine knee analog for validation of its functionality. Cadaver testing is to be conducted with an optimized prototype.

### Example 3: Human Cadaver Test of Prototype 3

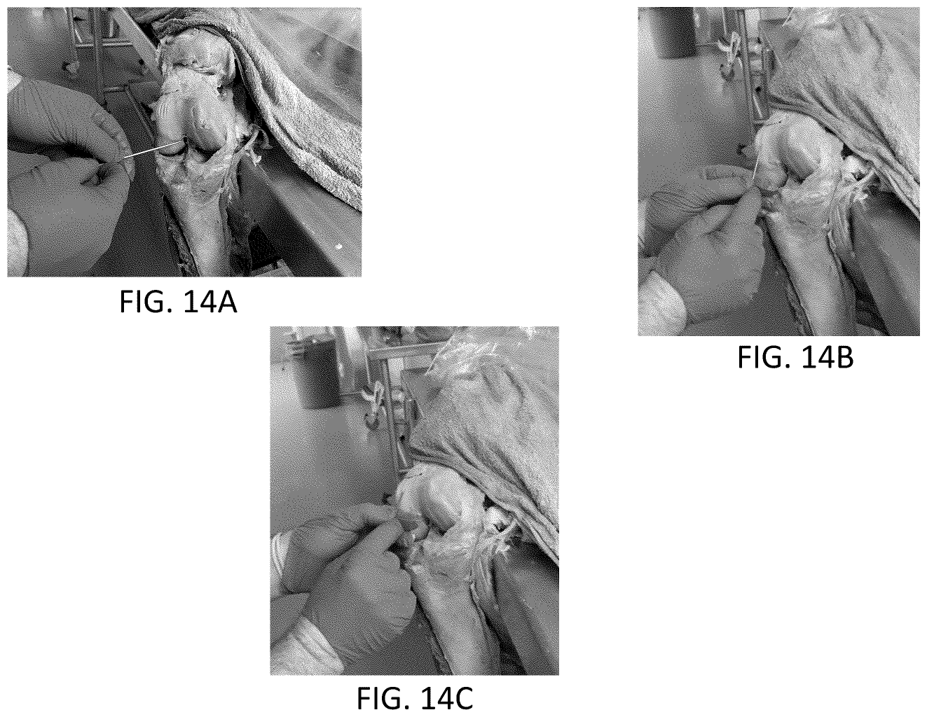

Gouge 301 of Device 300 (Prototype 3) is tested on an open human cadaver knee on the non-load bearing regions for validation of its excising functionality (FIG. 14A, 14B, 14C). Gouge 301 is capable of cutting the articular cartilage. Further cadaver testing is to be conducted with the gouge 301 and gripper 302 to tests cutting and harvesting abilities. Cadaver testing of whole device 300 is to be conducted with an optimized prototype.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A device for harvesting cartilage from a subject, wherein the device is configured to excise and harvest cartilage from the subject in a minimally invasive manner.

2. The device of claim 1, wherein the device is configured to be inserted into a subject via a cannula.

3. The device of claim 2, wherein the device is configured to be inserted into a knee or a shoulder of the subject.

4. The device of claim 3, wherein the device comprises
a lower gouge configured to excise cartilage away from a cartilage surface, and
an upper jaw that rotates about the lower gouge, wherein the upper jaw is configured to close down and grip the excised cartilage.

5. The device of claim 4, wherein the device is configured to retract the harvested cartilage back into the cannula for extraction.

6. The device of claim 5, wherein the device is configured to be completely contained within the cannula.

7. The device of claim 6, wherein the cannula has an outer diameter of about 2.77 mm.

8. The device of claim 3, wherein the device is configured to be actuated by a handle with a sliding thumb grip.

9. The device of claim 3, wherein the device is capable of providing about 200 mg of cartilage in 3-5 passes.

10. The device of claim 1, wherein the cartilage is articular cartilage from either the medial or lateral femoral condyle or the intercondylar notch.

11. A method of harvesting cartilage from a subject, the method comprising
inserting into a knee or shoulder of a subject a device comprising a lower gouge configured to excise cartilage away from a cartilage surface, and an upper jaw which rotates about the lower gouge, wherein the upper jaw is configured to close down and grip the excised cartilage; and
excising and harvesting a cartilage from the knee or shoulder of the subject in a single pass by actuating the device.

12. The method of claim 11, wherein the cartilage is articular cartilage.

13. The method of claim 11, wherein the cartilage is from the non load-bearing surfaces of the medial and lateral femoral condyles.

14. The method of claim 11, wherein the cartilage is harvested for an autologous chondrocyte implantation procedure.

15. The method of claim 14, wherein about 200 mg of cartilage is harvested in 3-5 passes.

16. The method of claim 11, wherein the cartilage is harvested for a clinical articular cartilage biopsy procedure.

17. The method of claim 11, wherein the device is inserted into the knee or shoulder of the subject via a cannula.

18. The method of claim 17, the method further comprising retracting the excised cartilage back into the cannula for extraction.

19. The method of claim 11, wherein the method further comprises administering local anesthesia to the subject.

20. The method of claim 11, wherein the cartilage is harvested without an arthroscopic surgery.
